SEQUENCE LISTING (6 sequences)

SEQ ID NO: 1 - protein, 1116 aa, Artificial Sequence
  PERK polypeptide
MERAISPGLLVRALLLLLLLLGLAARTVAAGRARGLPAPTAEAAFGLGAAAAPTSATRVPAAGAVAAAEVTVEDAEALPAAAGEQEPRGPEPDDETELRPRGRSLVIISTLDGRIAALDPENHGKKQWDLDVGSGSLVSSSLSKPEVFGNKMIIPSLDGALFQWDQDRESMETVPFTVESLLESSYKFGDDVVLVGGKSLTTYGLSAYSGKVRYICSALGCRQWDSDEMEQEEDILLLQRTQKTVRAVGPRSGNEKWNFSVGHFELRYIPDMETRAGFIESTFKPNENTEESKIISDVEEQEAAIMDIVIKVSVADWKVMAFSKKGGHLEWEYQFCTPIASAWLLKDGKVIPISLFDDTSYTSNDDVLEDEEDIVEAARGATENSVYLGMYRGQLYLQSSVRISEKFPSSPKALESVTNENAIIPLPTIKWKPLIHSPSRTPVLVGSDEFDKCLSNDKFSHEEYSNGALSILQYPYDNGYYLPYYKRERNKRSTQITVRFLDNPHYNKNIRKKDPVLLLHWWKEIVATILFCIIATTFIVRRLFHPHPHRQRKESETQCQTENKYDSVSGEANDSSWNDIKNSGYISRYLTDFEPIQCLGRGGFGVVFEAKNKVDDCNYAIKRIRLPNRELAREKVMREVKALAKLEHPGIVRYFNAWLEAPPEKWQEKMDEIWLKDESTDWPLSSPSPMDAPSVKIRRMDPFATKEHIEIIAPSPQRSRSFSVGISCDQTSSSESQFSPLEFSGMDHEDISESVDAAYNLQDSCLTDCDVEDGTMDGNDEGHSFELCPSEASPYVRSRERTSSSIVFEDSGCDNASSKEEPKTNRLHIGNHCANKLTAFKPTSSKSSSEATLSISPPRPTTLSLDLTKNTTEKLQPSSPKVYLYIQMQLCRKENLKDWMNGRCTIEERERSVCLHIFLQIAEAVEFLHSKGLMHRDLKPSNIFFTMDDVVKVGDFGLVTAMDQDEEEQTVLTPMPAYARHTGQVGTKLYMSPEQIHGNSYSHKVDIFSLGLILFELLYPFSTQMERVRTLTDVRNLKFPPLFTQKYPCEYVMVQDMLSPSPMERPEAINIIENAVFEDLDFPGKTVLRQRSRSLSSSGTKHSRQSNNSHSPLPSN

SEQ ID NO: 2 - DNA, 21 bp, Artificial Sequence
  Sense strand
  misc_RNA (1)..(19): ribonucleotides
  misc_feature (20)..(21): deoxyribonucleotides
gcaugcagucucagacccatt

SEQ ID NO: 3 - DNA, 21 bp, Artificial Sequence
  Antisense strand
  misc_RNA (1)..(19): ribonucleotides
  misc_feature (20)..(21): deoxyribonucleotides
ugggucugagacugcaugctt

SEQ ID NO: 4 - DNA, 22 bp, Artificial Sequence
  Synthetic oligonucleotide forward primer
tctgacaagacgttcccaatca

SEQ ID NO: 5 - DNA, 22 bp, Artificial Sequence
  Synthetic oligonucleotide reverse primer
gaataacttccctccgaccaca

SEQ ID NO: 6 - DNA, 28 bp, Artificial Sequence
  Synthetic oligonucleotide probe
tgttggaagggaagataaacggctacgc

(19) United States
(12) Patent Application Publication
(10) Pub. No.: US 2022/0202849 A1
(43) Pub. Date: Jun. 30, 2022

(54) PERK AS A TARGET FOR VIRUS THERAPEUTICS

(71) Applicant: George Mason University, Fairfax, VA (US)

(72) Inventors: Kylene Wesley KEHN-HALL, Blacksburg, VA (US); Bibha DAHAL, Silver Spring, MD (US); Jonathan DINMAN, Potomac, MD (US); Jonathan Larson Jacobs, Rockville, MD (US)

(73) Assignee: George Mason University, Fairfax, VA (US)

(21) Appl. No.: 17/497,187

(22) Filed: Oct. 8, 2021

Related U.S. Application Data
(60) Provisional application No. 63/089,669, filed on Oct. 9, 2020.

(51) Int. Cl. A61K 31/7105; A61P 31/12

(52) U.S. Cl. CPC A61K 31/7105; A61P 31/12

(57) ABSTRACT

The present disclosure provides compositions and methods for modulating PERK activity and/or expression in cells for use in treatment or prevention of viral infection, including by reducing translation of viral non-structural proteins in cells using compositions and/or compounds that inhibit PERK activity and/or expression.

### CROSS-REFERENCE TO RELATED APPLICATIONS

This application claims the benefit of U.S. Provisional Patent Application Ser. No. 63/089,669 (filed on Oct. 9, 2020), the disclosure of which is incorporated herein by reference in its complete entirety.

### GOVERNMENT LICENSE RIGHTS

This invention was made with government support under Grant No. HDTRA1-18-1-0045 awarded by the Defense Threat Reduction Agency. The government has certain rights in the invention.

### REFERENCE TO SEQUENCE LISTING

The Sequence Listing concurrently submitted herewith as a text file named “Sequence_Listing_ST25.txt,” created on Oct. 7, 2020, and having a size of 11,244 bytes is herein incorporated by reference pursuant to 37 C.F.R. § 1.52(e)(5).

### TECHNICAL FIELD

One or more embodiments set forth herein relate to compositions and methods for modulating protein kinase R (PKR)-like endoplasmic reticulum kinase (PERK) activity and/or expression in cells for use in treatment or prevention of viral infection.

### BACKGROUND

Viruses including members of the Togaviridae family, genera alphaviruses pose a serious health threat. For example, Venezuelan equine encephalitis virus (VEEV) and eastern equine encephalitis virus (EEEV) are alphaviruses that cause significant disease and sometimes death in humans and equines (horses, donkeys). In some cases, infection progresses to the brain and encephalitis occurs. Long-term neurological deficits are often seen in survivors that have suffered from encephalitis. Similarly, viruses including members of the Bunyaviridae, genus Phlebovirus, and members of the Flaviviridae, genus Flavivirus cause significant disease in humans and animals. Studies targeting viral proteins as potential therapeutics have been reported. Unfortunately, there are no FDA approved therapeutics available to treat VEEV, EEEV, RVFV, and ZIKV induced diseases.

There is a need for new and effective antiviral agents as well as therapeutic and/or prophylactic methods and strategies that target viruses.

### SUMMARY

In one aspect, the present disclosure provides a method for reducing translation of a non-structural protein of a virus in a cell. The method comprises one or more of the following: contacting the cell with an effective amount of a composition that inhibits PERK activity and/or expression in the cell, thereby reducing translation of the non-structural protein.

In another aspect, the present disclosure provides a method for treating or preventing infection with a virus. The method comprises one or more of the following: administering to a subject an effective amount of a composition that inhibits PERK activity and/or expression in a cell of the subject, thereby reducing translation of a nonstructural protein of the virus in the cell.

In some aspects, the present disclosure provides a method for enhancing viability of a cell infected with a virus. The method comprises one or more of the following: contacting the cell with an effective amount of a composition that inhibits PERK activity and/or expression in the cell, thereby preventing virus-induced apoptosis in the cell.

In other aspects, the present disclosure provides a composition comprising an effective amount of a compound that inhibits PERK activity and/or expression in a cell infected with a virus, wherein the effective amount is sufficient to reduce translation of a nonstructural protein of the virus in the cell.

In one example embodiment, a composition comprises an effective amount of a compound that inhibits PERK activity and/or expression in a cell infected with a virus, wherein the effective amount is sufficient to reduce translation of a nonstructural protein of the virus in the cell.

### DRAWINGS

The various advantages of the examples of the present disclosure will become apparent to one skilled in the art by reading the following specification and appended claims, and by referencing the following drawings, in which:

FIGS. 1A through 1E illustrate graphs and gel showing siRNA knockdown of PERK decreases VEEV replication in primary astrocytes. Primary human astrocytes were transfected with 100 nM of siNeg or siPERK siRNAs. At 48 h post transfection, cells were mock infected or infected with VEEV-TC83 (MOI 5). A) Viral replication was analyzed via plaque assays in Vero cells using supernatants collected at 18 hpi. B) Cell lysates were collected at 18 hpi and analyzed by immunoblot. PVDF membranes were probed for levels of PERK, VEEV nsP2, VEEV glycoprotein (GP), and VEEV capsid. β-Actin was used as a loading control. C-D) Data show the quantitation of the respective immunoblots. Protein levels expressed in each blot were normalized to β-actin and normalized values were calculated relative to siNeg transfected cells. N=3, *p≤0.05, ***p≤0.001, ****p≤0.0001. E) Cell viability was measured using CellTiter-Glo assay at 48 hpi. Data was normalized to siNeg-transfected and mock-infected cells. Data are expressed as the Mean±SD (n=4). ****p≤0.0001.

FIGS. 2A through 2D illustrate graphs and gel showing siRNA knockdown of PERK on VEEV replication in U87MG cells. U87MG cells were transfected with 100 nM of siNeg or siPERK siRNAs. At 48 h post transfection, cells were mock infected or infected with VEEV-TC83 (MOI 5). A) Viral replication was analyzed via plaque assays in Vero cells using supernatants collected from U87MG cells at 16 hpi. B) Cell lysates were collected at 16 hpi and analyzed by immunoblot. PVDF membranes were probed for levels of PERK, VEEV nsP2, VEEV GP, and VEEV capsid. β-Actin was used as a loading control. C-D) Data show the quantitation of the respective immunoblots. Protein levels expressed in each blot were normalized to β-actin and normalized values were calculated relative to siNeg transfected cells. N=3, ***p≤0.001.

FIGS. 3A through 3C illustrate graphs and gel showing loss of PERK decreases VEEV titers in pericytes and HUVECs. A) Pericytes or HUVECs were transfected with 100 nM of siNeg or siPERK siRNAs. Cell lysates were collected 48 hours post-transfection and analyzed by immunoblot. PVDF membranes were probed for levels of PERK. β-actin was used as a loading control. B) Quantitative data of panel A. PERK protein levels were normalized to β-actin and normalized values were calculated relative to siNeg transfected cells. C) At 48 h post transfection, cells were infected with VEEV TC83 (MOI 5) and viral replication was analyzed using supernatants collected at 18 hpi via plaque assays in Vero cells. B) Data are expressed as the Mean±SD (n=3). *p≤0.05, **p≤0.01.

FIGS. 4A and 4B illustrate graphs showing siRNA knockdown of PERK impact on viral replication in New World Alphaviruses. Primary astrocytes were transfected with 100 nM of siNeg or siPERK siRNAs. At 48 h post transfection, cells were infected with A) VEEV TrD (MOI 0.5) or B) EEEV FL93-939 (MOI 0.5). Viral replication was analyzed using supernatants collected at 18 hpi via plaque assays in Vero cells. Data are expressed as the Mean±SD (n=3). *p≤0.05.

FIGS. 5A and 5B illustrate graphs showing PERK impact on early events of VEEV replication. A-B) Primary astrocytes were transfected with 100 nM of siNeg or siPERK siRNAs prior to VEEV TC83 infection (MOI of 5). A) Supernatants were collected at the indicated time points and viral titers determined via plaque assay. B) Cell lysates were collected at the indicated time points. RNA extraction was performed, and viral genomic copies determined by RT-qPCR. Data are expressed as the Mean±SD (n=3). *p≤0.05, **p≤0.01, ***p≤0.001, ****p≤0.0001.

FIGS. 6A and 6B illustrate graphs showing VEEV dsRNA is not synthesized in cells lacking PERK. Primary astrocytes were transfected with 100 nM of siNeg or siPERK siRNAs. At 48 h post transfection, A) Cells were infected with VEEV TC-83 (MOI 5). Total RNA was isolated at 3, 6, 9, 12 hpi, and 18 hpi. IFN-β gene expression was determined by RT-qPCR. Fold changes were calculated relative to 18S ribosomal RNA and normalized to mock samples using the ΔΔCt method. Data are expressed as the Mean±SD (n=3). B) Cells were treated with poly (I:C) complexed with Lipofectamine 2000 for 6 hours. siNeg transfected cells treated with Lipofectamine only was used as a control. IFN-β gene expression determined as described above. Data are expressed as the Mean±SD (n=3). *p≤0.5, ***p≤0.001.

FIGS. 7A through 7D illustrate graphs showing loss of PERK signaling inhibits translation of incoming alphavirus genomes. A) Primary astrocytes were transfected with 100 nM of siNeg or siPERK siRNAs. At 48 h post transfection, cells were infected with VEEV (MOI 5), and RNA collected 1 hpi. Viral genomic copies were determined by RT-qPCR. Results are displayed as genomic copies in logarithmic scale. Data are expressed as the Mean±SD (n=3). *p≤0.05. B-C) Primary astrocytes were transfected with 100 nM of siNeg or siPERK siRNAs. At 48 h post transfection, cells were infected with VEEV nsP3-nLuc (panel B) or EEEV nsP3-nLuc at MOI of 5 (panel C). At 18 hpi, luminescence was measured using Promega's Nano-Glo Luciferase Assay system. Data are expressed as the Mean±SD (n=5). ***p≤0.001, ****p≤0.0001. D) Transfected cells were electroporated with translation reporter RNAs. Cells were lysed 2 h post electroporation and luciferase activity was measured. Luminescence (relative luminescence units per μg protein) is shown expressed as a fold change over siNeg transfected and VEEV reporter RNA electroporated cells. Data are expressed as the Mean±SD (n=3). ***p≤0.001.

FIGS. 8A and 8B illustrate graphs showing loss of PERK results in reduction of Rift Valley fever virus (RVFV) and Zika virus (ZIKV) titers. Primary astrocytes were transfected with 100 nM of siNeg or siPERK siRNAs. At post 48 h post transfection, cells were infected with A) RVFV MP12 (MOI 5) or B) ZIKV MR776 (MOI 5). Supernatants were collected at the indicated time points and viral titers determined via plaque assay. Data are expressed as the Mean±SD (n=3). *p≤0.5, **p≤0.02.

### DESCRIPTION

One or more embodiments set forth, described, and/or illustrated herein is based at least on a surprising discovery that PERK, a host protein, is required for alphavirus replication, in particular, for translation of non-structural protein(s), and that cells that are depleted in PERK are unable to support viral replication, indicating that targeting PERK is a therapeutic and/or prophylactic strategy for treatment and/or prevention of viral infection and virus-induced disease. One or more embodiments set forth, described, and/or illustrated herein include methods, compositions, and kits related to modulating PERK activity and/or expression in the cell, thereby reducing translation of the non-structural protein(s) of the virus, and strategies for leveraging the therapeutic and/or prophylactic potential thereof. These surprising discoveries involving targeting the host protein PERK appear to be paradoxical when put in the context of the traditional role of PERK in suppressing translation and run counter to the conventional wisdom of antivirals targeting viral proteins.

In one example embodiment, a method for reducing translation of a non-structural protein of a virus in a cell comprises: contacting the cell with an effective amount of a composition that inhibits PERK activity and/or expression in the cell, thereby reducing translation of the non-structural protein.

The terms “reducing translation” and “reduce translation,” as used herein in the context of a non-structural protein of a virus in a cell, is intended to refer to reducing the amount of translation as well as preventing translation of the non-structural protein in the cell.

In one example embodiment, the virus is an alphavirus. Generally, alphavirus virions are enveloped with viral glycoproteins, E1 & E2, incorporated into the membrane. The genome is approximately 11.4 kb and is positive sense single stranded RNA encoding two open reading frames. Four non-structural proteins (nsP1-4) are encoded by the first reading frame which begins at the 5′ end of the genome. These four different non-structural proteins can function during infection as polyproteins as well as cleaved final products. Without wishing to be bound by any particular theory, it is believed that the replicase activity resides in a complex of nonstructural proteins and host proteins. The subgenomic reading frame encodes for the structural proteins including capsid and three envelope proteins (E2, 6K, and E1). A small E3 protein is also encoded but not incorporated in the virion.

The alphavirus of this disclosure can be any alphavirus in the family Togaviridae, preferably any alphavirus against which it is desirable to elicit an immune response in a subject. Nonlimiting examples of an alphavirus of this disclosure include Venezuelan equine encephalitis virus (VEEV), Eastern equine encephalitis virus (EEEV), Western equine encephalitis virus (WEEV), Sindbis virus (SINV), Chikungunya virus (CHIKV), O'Nyong-Nyong virus (ONNV), Ross River virus (RRV), Mayaro virus (MAW), Semliki Forest virus (SFV), Barmah Forest virus, Everglades, Mucambo, Pixuna, Middelburg, Getah, Bebaru, Una, Okelbo, Babanki, Fort Morgan, Ndumu and subgroups and strains thereof.

Examples of viruses classified as New World alphaviruses include, but are not limited to, VEEV, EEEV, and WEEV; and examples of viruses classified as Old World alphaviruses include, but are not limited to, SINV, CHIKV, ONNV, RRV, MAW, and SFV.

In one example embodiment, the alphavirus is a VEEV, an EEEV, a WEEV, a SINV, a CHIKV, a ONNV, a RRV, a MAW, or an SFV.

In another example embodiment, the alphavirus is a VEEV, an EEEV, or a WEEV. In other example embodiments, the alphavirus is a SINV or a CHIKV.

In some example embodiments, the alphavirus is a VEEV.

In one example embodiment, the virus is an alphavirus, wherein the effective amount of the composition inhibits the PERK activity and/or expression in the cell, thereby reducing translation of nsP1, nsP2, nsP3, and/or nsP4 in the cell.

In another example embodiment, the virus is a Flavivirus. Flaviviruses contain a positive-sense genomic RNA with a 5′cap. Upon viral entry, viral translation occurs through a canonical dependent process, similar to alphaviruses.

In an example embodiment, the Flavivirus is a mosquito-borne virus. Nonlimiting examples of mosquito-borne flaviviruses of this disclosure include zika virus (ZIKV), denge virus (DENV), West Nile virus (WNV), yellow fever virus (YFV), and Japanese encephalitis virus (JEV).

In an example embodiment, the Flavivirus is a ZIKV.

In another example embodiment, the virus is a negative sense RNA virus, such as RVFV of the genus Phlebovirus. Negative-sense RNA viruses generally require transcription of their genetic material before translation can occur. Additionally, RVFV mRNAs acquire their 5′ caps through cap-snatching from cellular mRNAs.

The cell can be any vertebrate cell capable of being infected with a virus.

In one example embodiment, the cell is a eukaryotic cell. In another example embodiment, the cell is a mammalian cell.

In another example embodiment, the cell is a neuron, a fibroblast, a lung epithelial cell, or a lymphocyte.

In some example embodiments, the cell is a brain cell.

In one example embodiment, the cell is a glial cell.

In other example embodiments, the cell is an astrocyte, an oligodendrocyte, or a Schwan cell.

In one example embodiment, the neuron is a cholinergic neuron, a GABAergic neuron, a glutamatergic neuron, a dopaminergic neuron, or a serotonergic neuron.

In another example embodiment, the cell is inside a subject (e.g., in a vertebrate, for example and without limitation, a human or animal).

In some example embodiments, the cell is an isolated cell.

In one example embodiment, the cell is in culture.

In some example embodiments, the disclosure includes compositions and methods for modulating (e.g., inhibiting) PERK expression (e.g., protein and/or nucleic acid (mRNA) expression) and/or activity (e.g., protein activity). Such compositions and methods generally include targeting (e.g., specifically targeting) PERK DNA, mRNA, and/or protein to thereby modulate (e.g., inhibit) PERK mRNA and/or protein expression and/or function.

In some example embodiments, targeting PERK can include targeting (e.g., specifically targeting) PERK in a cell infected with the virus.

In other example embodiments, targeting PERK can include targeting (e.g., specifically targeting) PERK in a cell susceptible to or at risk of infection with the virus.

PERK set forth, described, and/or illustrated herein includes PERK DNA, mRNA, and/or protein, including full length transcripts and proteins, truncated transcripts, and proteins (e.g., truncated PERK transcripts and proteins that exhibit or have detectable PERK activity), and/or mutant or mutated PERK transcripts and protein, truncated or otherwise (e.g., that exhibit or have detectable PERK activity).

In some example embodiments, the PERK comprises the amino acid sequence as disclosed by any one of GenBank Accession Nos. NM_004836.7, AF193339.1, AF110146.1, NM_001313915.1, CR749382.1, BC126354.1, BC126356.1, and AK315287.1, which are herein incorporated by reference in their entirety, or a sequence variant thereof.

In one example embodiment, the PERK is a protein comprising the amino acid sequence as set forth in SEQ ID NO:1, a sequence variant thereof, or a polynucleotide sequence having at least about 50% sequence identity thereto, illustratively, at least about 50%, 55%, 60%, 65%, 70%, 75%, 80%, 85%, 90%, 91%, 92%, 93%, 94%, 95%, 96%, 97%, 98%, or 99% sequence identity or 100% sequence identity thereto:

(SEQ ID NO: 1)
MERAISPGLLVRALLLLLLLLGLAARTVAAGRARGLPAPTAEAAFGLGA
AAAPTSATRVPAAGAVAAAEVTVEDAEALPAAAGEQEPRGPEPDDETEL
RPRGRSLVIISTLDGRIAALDPENHGKKQWDLDVGSGSLVSSSLSKPEV
FGNKMIIPSLDGALFQWDQDRESMETVPFTVESLLESSYKFGDDVVLVG
GKSLTTYGLSAYSGKVRYICSALGCRQWDSDEMEQEEDILLLQRTQKTV
RAVGPRSGNEKWNFSVGHFELRYIPDMETRAGFIESTFKPNENTEESKI
ISDVEEQEAAIMDIVIKVSVADWKVMAFSKKGGHLEWEYQFCTPIASAW
LLKDGKVIPISLFDDTSYTSNDDVLEDEEDIVEAARGATENSVYLGMYR
GQLYLQSSVRISEKFPSSPKALESVTNENAIIPLPTIKWKPLIHSPSRT
PVLVGSDEFDKCLSNDKFSHEEYSNGALSILQYPYDNGYYLPYYKRERN
KRSTQITVRFLDNPHYNKNIRKKDPVLLLHWWKEIVATILFCIIATTFI
VRRLFHPHPHRQRKESETQCQTENKYDSVSGEANDSSWNDIKNSGYISR
YLTDFEPIQCLGRGGFGVVFEAKNKVDDCNYAIKRIRLPNRELAREKVM
REVKALAKLEHPGIVRYFNAWLEAPPEKWQEKMDEIWLKDESTDWPLSS
PSPMDAPSVKIRRMDPFATKEHIEIIAPSPQRSRSFSVGISCDQTSSSE
SQFSPLEFSGMDHEDISESVDAAYNLQDSCLTDCDVEDGTMDGNDEGHS
FELCPSEASPYVRSRERTSSSIVFEDSGCDNASSKEEPKTNRLHIGNHC
ANKLTAFKPTSSKSSSEATLSISPPRPTTLSLDLTKNTTEKLQPSSPKV
YLYIQMQLCRKENLKDWMNGRCTIEERERSVCLHIFLQIAEAVEFLHSK
GLMHRDLKPSNIFFTMDDVVKVGDFGLVTAMDQDEEEQTVLTPMPAYAR
HTGQVGTKLYMSPEQIHGNSYSHKVDIFSLGLILFELLYPFSTQMERVR
TLTDVRNLKFPPLFTQKYPCEYVMVQDMLSPSPMERPEAINIIENAVFE
DLDFPGKTVLRQRSRSLSSSGTKHSRQSNNSHSPLPSN (GenBank
Accession Nos. NM_004836.7 (Homo sapiens)).

Variants include a homologous PERK protein encoded by the same genetic locus in an organism, i.e., an allelic variant. Variants also encompass proteins derived from other genetic loci in an organism but having substantial homology to e.g., PERK having the sequence of SEQ ID NO: 1. Variants also include proteins homologous to PERK having the sequence of e.g., SEQ ID NO: 1 but in another organism, e.g., an ortholog.

Compositions for modulating PERK expression and/or activity can include, but are not limited to, one or more of inhibitory nucleic acids, small molecules, antibodies, and inhibitory peptides. For example, one or more of an inhibitory nucleic acid, a small molecule, an anti-PERK antibody, and/or an inhibitory peptide can be used to target (e.g., specifically target) PERK (e.g., SEQ ID NO:1 or a variant thereof) in a cell, thereby modulating (e.g., inhibiting) PERK to reduce translation of a non-structural protein of a virus in a cell infected with the virus.

Inhibitory nucleic acids suitable for use in the methods set forth, described, and/or illustrated herein include inhibitory nucleic acids that bind (e.g., bind specifically) to PERK. Also encompassed are inhibitory nucleic acids that bind (e.g., bind specifically) to a component of the PERK signaling pathway upstream or downstream of PERK. Exemplary inhibitory nucleic acids include, but are not limited to, siRNA and antisense nucleic acids. For example, the disclosure includes siRNA and antisense nucleic acids that target or bind (e.g., specifically target or specifically bind) to PERK mRNA in a cell, thereby modulating (e.g., inhibiting) PERK to reduce translation of a non-structural protein of a virus in a cell infected with the virus.

RNA interference (RNAi) is a method of post-transcriptional gene regulation that is conserved throughout many organisms. RNAi can be induced by short (e.g., <30 nucleotide) double stranded RNA (“dsRNA”) molecules. These short dsRNA molecules, called “short interfering RNA” or “siRNA,” cause the destruction of messenger RNAs (“mRNAs”) which share sequence homology with the siRNA to within one nucleotide resolution. It is believed that the siRNA and the targeted mRNA bind to an RNA-induced silencing complex (RISC), which cleaves the targeted mRNA. For example, in mammalian cells, RNAi can be triggered by short (e.g., 21 nucleotide) duplexes of small interfering RNA (siRNA), by micro-RNAs (miRNA), functional small-hairpin RNA (shRNA), and/or other dsRNAs which are expressed in vivo e.g., using DNA templates with RNA polymerase III promoters.

RNAi useful for inhibiting PERK can include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can be transcribed in vitro from a DNA template, or in vivo from, e.g., shRNA. The dsRNA molecules can be designed using any method known in the art and a number of algorithms are known and commercially available. Gene walk methods known in the art can be used to optimize the inhibitory activity of the siRNA.

In some example embodiments, a consensus sequence of a PERK gene can be used to design and generate RNAi. Synthesized RNA silencing constructs can be introduced into vectors designed for cell transformation. Different promoters can be used to drive expression of RNA silencing constructs such as constitutive promoters of different strength and origin.

In some example embodiments, a PERK gene regulatory region (e.g., promoter) may also be employed to direct expression of the silencing construct within the same cells and tissues as the endogenous gene.

In some example embodiments, a PERK mRNA may contain target sequences in common with their respective alternative splice forms, cognates, or mutants. A single siRNA comprising such a common targeting sequence can therefore induce RNAi-mediated degradation of different RNA types which contain the common targeting sequence.

In some example embodiments, the siRNA as set forth, described, and/or illustrated herein can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

In another example embodiment, an siRNA as set forth, described, and/or illustrated herein is an “isolated” siRNA e.g., altered or removed from the natural state through human intervention. For example, an siRNA naturally present in a living animal is not “isolated,” but a synthetic siRNA, or an siRNA partially or completely separated from the coexisting materials of its natural state is “isolated.”

In some example embodiments, the isolated siRNA can exist in substantially purified form, or can exist in a non-native environment such as, for example, a cell into which the siRNA has been delivered.

In other example embodiments, one or both strands of the siRNA of the disclosure can also comprise a 3′ overhang e.g., at least one unpaired nucleotide extending from the 3′-end of a duplexed RNA strand.

In some example embodiments, an siRNA of the disclosure comprises at least one 3′ overhang of from 1 to about 6 nucleotides (which includes ribonucleotides or deoxynucleotides) in length, preferably from 1 to about 5 nucleotides in length, more preferably from 1 to about 4 nucleotides in length, and particularly preferably from about 2 to about 4 nucleotides in length.

In one example embodiment, in which both strands of the siRNA molecule comprise a 3′ overhang, the length of the overhangs can be the same or different for each strand.

In some example embodiments, each strand of the siRNA of the disclosure can comprise 3′ overhangs of dithymidylic acid (“TT”) or diuridylic acid (“uu”).

The siRNA as set forth, described, and/or illustrated herein can be targeted to any stretch of approximately 19-25 contiguous nucleotides in a target PERK mRNA sequence. Techniques for selecting target sequences for siRNA are described, for example, Pei, Y. & Tuschl, T., On the art of identifying effective and specific siRNAs, Nat. Methods 3:670-676 (2006), Meister, G. & Tuschl, T., Mechanisms of gene silencing by double-stranded RNA, Nature 431:343-349 (2004), Dorsett, Y. & Tuschl, T., siRNAs: applications in functional genomics and potential as therapeutics, Nat. Rev. Drug Discov., 3, 318-329 (2004), which are herein incorporated by reference in their entireties.

In some example embodiments, the sense strand of the siRNA comprises a nucleotide sequence identical to any contiguous stretch of about 19 to about 25 nucleotides in the target PERK mRNA.

In other example embodiments, a target sequence on the target PERK mRNA can be selected from a given cDNA sequence corresponding to the target PERK mRNA, for example and without limitation, beginning 50 to 100 nt downstream (i.e., in the 3′ direction) from the start codon.

In some example embodiments, the target sequence can be in the 5′ or 3′ untranslated regions, or in the region nearby the start codon.

In some example embodiments, the agent is an siRNA that targets a PERK.

In other example embodiments, the siRNA that targets the PERK is obtained from a commercially available source e.g., SignalSilence® PERK siRNA I (Cat # 9024S) (Cell Signaling Technology, Inc., Danvers, Mass.).

In one example embodiment, the siRNA is synthesized to have the following targeting sequences: PERK (GenBank Accession Nos. NM_004836.7, EIF2AK3, targeting sequence: sense GCAUGCAGUCUCAGACCCAtt (SEQ ID NO:2) and antisense UGGGUCUGAGACUGCAUGCtt (SEQ ID NO:3).

siRNA can be delivered into cells by methods known in the art, e.g., cationic liposome transfection and electroporation. Direct delivery of siRNA in saline or other excipients can silence target genes in various tissues. Liposomes and nanoparticles can also be used to deliver siRNA. Delivery methods using liposomes, e.g., stable nucleic acid-lipid particles (SNALPs), dioleoyl phosphatidylcholine (DOPC)-based delivery system, as well as lipoplexes, e.g., lipofectamine 2000 (Thermofisher, Cat# 11668030). Conjugating siRNA to peptides, RNA aptamers, antibodies, or polymers, e.g., dynamic polyconjugates, cyclodextrin-based nanoparticles, atelocollagen, and chitosan, can improve siRNA stability and/or uptake. Viral-mediated delivery mechanisms can also be used to induce specific silencing of targeted genes through expression of siRNA, for example, by generating recombinant adenoviruses harboring siRNA e.g., under RNA Pol II promoter transcription control.

siRNA duplexes can also be expressed within cells from engineered RNAi precursors, e.g., recombinant DNA constructs using mammalian Pol III promoter systems (e.g., HI or U6/snRNA promoter systems capable of expressing functional double-stranded siRNAs.

siRNA can also be expressed in a miRNA backbone which can be transcribed by either RNA Pol II or III. MicroRNAs are endogenous noncoding RNAs of approximately 22 nucleotides in cells that can post-transcriptionally regulate gene expression. One common feature of miRNAs is that they are excised from an approximately 70 nucleotide precursor RNA stem loop by Dicer, an RNase III enzyme, or a homolog thereof. By substituting the stem sequences of the miRNA precursor with the sequence complementary to the target mRNA, a vector construct can be designed to produce siRNAs to initiate RNAi against specific mRNA targets in mammalian cells. When expressed by DNA vectors containing polymerase II or III promoters, miRNA designed hairpins can silence gene expression.

Engineered RNA precursors, introduced into cells or whole organisms as described herein, can lead to the production of a desired siRNA molecule. Such an siRNA molecule will then associate with endogenous protein components of the RNAi pathway to bind to and target a specific mRNA sequence for cleavage, destabilization, and/or translation inhibition destruction. In this fashion, the mRNA (e.g., PERK mRNA) to be targeted by the siRNA generated from the engineered RNA precursor will be depleted from the cell or organism, leading to a decrease in the concentration of the protein encoded by that mRNA in the cell or organism.

Antisense oligonucleotides, when introduced into a target cell, can specifically bind to their target polynucleotide and interfere with transcription, RNA processing, transport, translation and/or stability. An “antisense” nucleic acid can include a nucleotide sequence that is complementary to a “sense” nucleic acid encoding a protein, e.g., complementary to the coding strand of a double-stranded cDNA molecule. In another example embodiment, the antisense nucleic acid molecule is antisense to a “noncoding region” of the coding strand of a nucleotide sequence (e.g., the 5′ and 3′ untranslated regions). An antisense nucleic acid can be constructed using chemical synthesis and enzymatic ligation reactions using procedures known in the art.

In other example embodiments, antisense and RNAi constructs, or DNA encoding such RNA's, may be employed to inhibit PERK gene transcription or translation or both within a cell, either in vitro or in vivo. In some example embodiments, such oligonucleotide may comprise any unique portion of a PERK nucleic acid sequence. In one example embodiment, such a sequence comprises at least 18, 19, 20, 21, 22, 23, 24, 25, 26, 27, 28, 29, or 30 or more contiguous nucleic acids of the nucleic acid sequence of a PERK, and/or complements thereof, which may be in sense and/or antisense orientation. In some example embodiments, by including sequences in both sense and antisense orientation, increased suppression of the corresponding PERK coding sequence may be achieved.

In other example embodiments, constructs may be designed that are complementary to all or part of the promoter and other control regions, exons, introns or even exon-intron boundaries of a PERK gene. In some example embodiments, constructs can include regions complementary to intron/exon splice junctions. In one example embodiment, a construct comprises complementarity to regions within 50-200 bases of an intron-exon splice junction.

In another example embodiment, an antisense nucleic acid can be designed such that it is complementary to the entire coding region of a target mRNA but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

Suitable small molecules include small molecules that inhibit PERK expression and/or activity directly, indirectly, or both directly and indirectly. Suitable small molecules include small molecules that bind (e.g., bind specifically) to PERK and thereby inhibit PERK expression and/or activity, and/or small molecules that do not bind to PERK or that bind to PERK with low affinity, but that inhibit PERK expression and/or activity by binding to a component of the PERK signaling pathway upstream or downstream of PERK.

In one example embodiment, the small molecule is an imidazolidinone compound or derivative that is an inhibitor of the activity of PERK as disclosed in e.g., International Publication No. WO 2017/046739, which is herein incorporated by reference for its disclosure of imidazolidinone derivatives that are inhibitors of PERK activity.

In another example embodiment, the small molecule is GSK2606414 (Catalog # 516535) (EMD Millipore, Cat # 516535), which is an orally available, potent, and selective PERK inhibitor with IC₅₀ of about 0.4 nM:

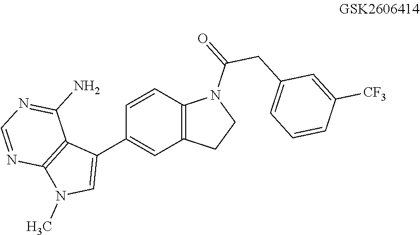 [SMILES: Cn1cc(-c2ccc3c(c2)CCN3C(=O)Cc2cccc(C(F)(F)F)c2)c2c(N)ncnc21]

In some example embodiments, the small molecule is GSK2656157 (Catalog # 5.04651.0001) (MilliporeSigma, Burlington, Mass.), which is a selective inhibitor of PERK with IC₅₀ of about 0.9 nM:

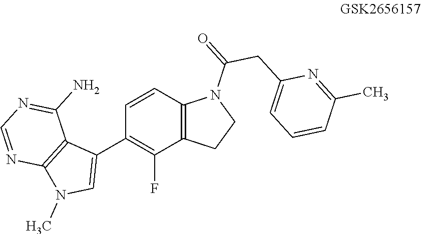 [SMILES: Cc1cccc(CC(=O)N2CCc3c2ccc(-c2cn(C)c4ncnc(N)c24)c3F)n1]

In other example embodiments, the small molecule is ISRIB (trans-isomer) (Catalog # 50-136-4741) (Fisher Scientific, Pittsburgh, Pa.), which is a potent and selective PERK inhibitor with IC₅₀ of about 5 nM:

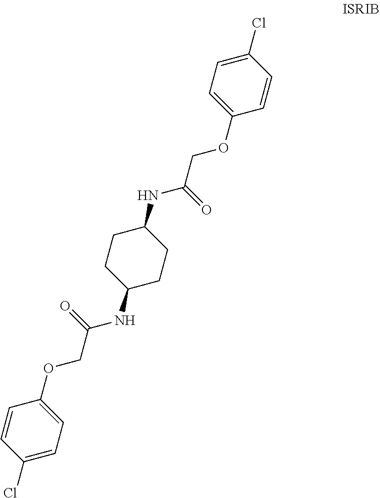 [SMILES: O=C(COc1ccc(Cl)cc1)N[C@H]1CC[C@@H](NC(=O)COc2ccc(Cl)cc2)CC1]

In other example embodiments, a Clustered Regularly Interspaced Short Palindromic Repeats (CRISPR)-CRISPR associated (Cas) (CRISPR/Cas) system may be used to inhibit PERK expression. A single Cas enzyme can be recruited to a specific DNA target (e.g., PERK DNA target) using a short RNA molecule that recognizes the specific DNA target. U.S. Pat. No. 8,697,359 is herein incorporated by reference for its disclosure of CRISPR-Cas systems and methods for altering expression of gene products.

For example, and without limitation, an engineered, non-naturally occurring vector system comprising one or more vectors can be introduced into a cell. The one or more vectors can comprise (a) a first regulatory element operably linked to one or more CRISPR/Cas system guide RNAs that hybridize with target sequences in genomic loci of the DNA molecules encoding the one or more gene products (e.g., PERK) and (b) a second regulatory element operably linked to a Type-II Cas9 protein. The components (a) and (b) can be located on same or different vectors of the system. The guide RNAs target the genomic loci of the DNA molecules encoding the one or more gene products and the Cas9 protein cleaves the genomic loci of the DNA molecules encoding the one or more gene products, whereby expression of the one or more gene products (e.g., PERK) is altered; and, wherein the Cas9 protein and the guide RNAs do not naturally occur together.

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes genes can be introduced into cells through gene-delivery mechanisms known in the art. A ribozyme having specificity for a target nucleic acid can include one or more sequences complementary to the nucleotide sequence of a cDNA described herein, and a sequence having known catalytic sequence responsible for mRNA cleavage. For example, a derivative of a Tetrahymena L-19 IVS RNA can be constructed in which the nucleotide sequence of the active site is complementary to the nucleotide sequence to be cleaved in a target mRNA. Alternatively, target mRNA can be used to select a catalytic RNA having a specific ribonuclease activity from a pool of RNA molecules.

In other example embodiments, ribozymes suitable for use in methods encompassed by the present disclosure include ribozymes that recognize and/or cleave PERK and/or components of the PERK signaling pathway upstream or downstream of PERK. For example, the disclosure includes ribozymes that recognize and/or cleave PERK mRNA, thereby modulating (e.g., inhibiting) PERK to reduce translation of a non-structural protein of a virus in a cell infected with the virus.

Aptamers are short oligonucleotide sequences which can specifically bind specific proteins. It has been demonstrated that different aptameric sequences can bind specifically to different proteins, and methods for selection and preparation of such aptamers are known in the art. Aptamers suitable for use in methods encompassed by the present disclosure include aptamers that bind (e.g., bind specifically) to PERK and/or components of the PERK signaling pathway upstream or downstream of PERK. In some example embodiments, the present disclosure provides aptamers that bind (e.g., bind specifically) to PERK amino acid sequences, thereby modulating (e.g., inhibiting) PERK to reduce translation of a non-structural protein of a virus in a cell infected with the virus.

One or more embodiments also include methods that include the use or administration of antibodies and antibody fragments that bind (e.g., bind specifically) to PERK and/or an epitope presented on native PERK and thereby inhibit PERK activity in a cell.

One or more embodiments further include methods that include the use or administration of inhibitory peptides that bind (e.g., bind specifically) to PERK or interact with PERK and thereby inhibit PERK activity and/or expression in a cell. Such peptides can bind or interact with an epitope on PERK and/or with a PERK domain. Suitable inhibitory peptides can that bind or interact with PERK can also be used to increase PERK degradation, for example, by increasing ubiquitination and/or proteosomal degradation of PERK.

Antibodies and inhibitory peptides can be modified to facilitate cellular uptake or increase in vivo stability. For example, acylation or PEGylation facilitates cellular uptake, increases bioavailability, increases blood circulation, alters pharmacokinetics, decreases immunogenicity and/or decreases the needed frequency of administration.

The term “effective amount,” as used herein, refers to an amount or a concentration of one or more compounds or a pharmaceutical composition described herein utilized for a period of time (including acute or chronic contact or administration and periodic or continuous contact or administration) that is effective within the context of its contact or administration for causing an intended effect or physiological outcome.

Effective amounts of one or more compounds or a pharmaceutical composition for use in the present disclosure include amounts that inhibit PERK expression, levels (e.g., protein levels) and/or activity (e.g., biological activity) in cells. In some example embodiments, a therapeutically or prophylactically effective amount of one or more compounds or a pharmaceutical composition described herein will provide, respectively, treatment or prevention of infection with the virus.

In other example embodiments, contacting the cell comprises contacting the cell in vivo in a subject comprising the cell. In some example embodiments, the subject is a vertebrate.

In one example embodiment, the subject can be a human or animal including livestock and companion animals. Companion animals include, for example and without limitation, animals kept as pets. Examples of companion animals include cats, dogs, and horses, as well as birds, such as parrots and parakeets. Livestock refers to animals reared or raised in an agricultural setting to make products such as food or fiber, or for its labor. In some example embodiments, livestock are suitable for consumption by mammals, for example humans. Examples of livestock animals include mammals, such as cattle, goats, horses, pigs, sheep, including lambs, and rabbits, as well as birds, such as chickens, ducks, and turkeys.

In some example embodiments, the subject is a human. In another example embodiment, the subject is a non-human mammal.

In other example embodiments, the subject can be a human who is a medical patient (e.g., a diabetes patient, or a patient in a hospital, clinic), a member of the armed services or law enforcement, a fire fighter, or a worker in the gas, oil, or chemical industry. In one example embodiment, the subject is an animal that is a veterinarian subject/patient (e.g., livestock or companion animal).

In one example embodiment, the subject is infected or at risk of infection with the virus.

In another example embodiment, the cell is a human or animal cell, and wherein contacting the cell comprises contacting the cell in vivo in a human or animal comprising the cell.

In some example embodiments, the composition is a pharmaceutical composition further comprising a pharmaceutically acceptable carrier.

In other example embodiments, the composition comprises a compound that inhibits the PERK activity and/or expression in the cell. In some example embodiments, the compound is a small interfering RNA (siRNA).

In other example embodiments, the method is for decreasing, inhibiting, and/or preventing virus replication in the cell.

In some example embodiments, the method is for decreasing, inhibiting, and/or preventing synthesis of dsRNA of the virus.

In one example embodiment, the method is for decreasing, inhibiting, and/or preventing viral RNA production.

In another example embodiment, the method is for decreasing, inhibiting, and/or preventing structural protein synthesis.

In some example embodiments, the method is for decreasing, inhibiting, and/or preventing production of infectious viral particles.

In one example embodiment, the method is for decreasing, inhibiting, and/or preventing production of viral capsid protein(s).

In another example embodiment, the method is for decreasing, inhibiting, and/or preventing production of viral glycoprotein(s).

In other aspects, the present disclosure provides a method for treating or preventing infection with a virus. The method comprises: administering to a subject an effective amount of a composition that inhibits PERK activity and/or expression in a cell of the subject, thereby reducing translation of a nonstructural protein of the virus in the cell.

In one example embodiment, the virus is as described herein. In another example embodiment, the virus is an alphavirus, wherein the effective amount of the composition inhibits the PERK activity and/or expression in the cell, thereby reducing translation of nsP1, nsP2, nsP3, and/or nsP4 in the cell.

In another example embodiment, the subject is a vertebrate. In another example embodiment, the vertebrate is a human or animal.

In some example embodiments, the subject is infected or at risk of infection with the virus.

In other example embodiments, the composition comprises a compound that inhibits the PERK activity and/or expression in the cell.

In another example embodiment, the compound is a small interfering RNA (siRNA).

In other example embodiments, virus replication in the cell is decreased, inhibited, and/or prevented.

In some example embodiments, synthesis of dsRNA of the virus is decreased, inhibited, and/or prevented.

In one example embodiment, viral RNA production is decreased, inhibited, and/or prevented.

In another example embodiment, structural protein synthesis is decreased, inhibited, and/or prevented.

In some example embodiments, production of infectious viral particles in cells is decreased, inhibited, and/or prevented.

In one example embodiment, production of viral capsid protein(s) in cells is decreased, inhibited, and/or prevented.

In another example embodiment, production of viral glycoprotein(s) in cells is decreased, inhibited, and/or prevented.

In one aspect, the present disclosure provides a method for enhancing viability of a cell infected with a virus. The method comprises contacting the cell with an effective amount of a composition that inhibits PERK activity and/or expression in the cell, thereby preventing virus-induced apoptosis in the cell.

In one example embodiment, the virus is as described herein. In another example embodiment, the virus is an alphavirus. In some example embodiments, the virus is Venezuelan equine encephalitis virus (VEEV). In other example embodiments, the non-structural protein is nsP1, nsP2, nsP3, and/or nsP4.

In another example embodiment, the subject is a vertebrate. In another example embodiment, the vertebrate is a human or animal.

In some example embodiments, the subject is infected or at risk of infection with the virus.

In other example embodiments, the composition comprises a compound that inhibits the PERK activity and/or expression in the cell.

In another example embodiment, the compound is a small interfering RNA (siRNA).

In one example embodiment, viral RNA production is decreased, inhibited, and/or prevented.

In another example embodiment, structural protein synthesis is decreased, inhibited, and/or prevented.

In some example embodiments, production of infectious viral particles in cells is decreased, inhibited, and/or prevented.

In one example embodiment, production of viral capsid protein(s) in cells is decreased, inhibited, and/or prevented.

In another example embodiment, production of viral glycoprotein(s) in cells is decreased, inhibited, and/or prevented.

In one example embodiment, the composition is pharmaceutical composition further comprising a pharmaceutically acceptable carrier.

In other aspects, pharmaceutical compositions of the present disclosure typically include a pharmaceutically acceptable carrier. For example, a pharmaceutically acceptable carrier includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include systemic and local routes of administration. Exemplary routes include, but are not limited to, parenteral, e.g., intravenous, intradermal, subcutaneous, oral (e.g., inhalation), transdermal (topical), and transmucosal, administration.

In some example embodiments, the compositions and/or compounds described herein may be formulated with pharmaceutically acceptable carriers or diluents as well as any other known adjuvants and excipients in accordance with conventional techniques such as those disclosed in e.g., Remington: The Science and Practice of Pharmacy, 19th Ed. (Easton, Pa.: Mack Publishing Company, 1995); Remington's Pharmaceutical Sciences, 18th Ed. (1990, Mack Publishing Co., Easton, Pa. 18042); Liberman, N. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

For example, compositions and/or compounds of the present disclosure can be formulated by admixture with one or more pharmaceutically acceptable, non-toxic excipients or carriers. Such formulations can In some example embodiments be used to treat or prevent infection with the virus, for example.

Common excipients for use in the formulations can include, without limitation, sterile water, saline, polyalkylene glycols such as polyethylene glycol, oils of vegetable origin, hydrogenated naphthalenes, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate). In some example embodiments, biocompatible, biodegradable lactide polymer, lactide/glycolide copolymer, polyoxyethylene-polyoxypropylene copolymers, or combinations thereof can be used as excipients for controlling the release of a peptide in vivo.

Compositions/formulations can be prepared for topical (e.g., transdermal, sublingual, ophthalmic, or intranasal) administration, parenteral administration (e.g., by subcutaneous, intrathecal, intraventricular, intramuscular, or intraperitoneal injection, or by intravenous drip, in the form of liquid solutions or suspensions in aqueous physiological buffer solutions), for oral administration (e.g., in the form of tablets or capsules), or for intranasal administration (e.g., in the form of powders, nasal drops, or aerosols), depending on whether local or systemic treatment is desired and on the area to be treated. Administration can be rapid (e.g., by injection) or can occur over a period of time (e.g., by slow infusion or administration of slow release formulations). Compositions for other routes of administration also can be prepared as desired using appropriate methods. In addition, compositions can be prepared for in vitro use.

Formulations for topical administration may include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Nasal sprays also can be useful, and can be administered by, for example, a nebulizer, an inhaler, or another nasal spray device. Conventional pharmaceutical carriers, aqueous, powder or oily bases, thickeners and the like may be useful.

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Compositions and formulations for parenteral, intrathecal, or intraventricular administration can include sterile aqueous solutions, which also can contain buffers, diluents and other suitable additives (e.g., penetration enhancers, carrier compounds and other pharmaceutically acceptable carriers).

In some example embodiments, pharmaceutical compositions can include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations can be useful for oral delivery of therapeutics due to their ease of formulation and efficacy of solubilization, absorption, and bioavailability.

Liposomes are vesicles that have a membrane formed from a lipophilic material and an aqueous interior that can contain the composition to be delivered. Liposomes can be particularly useful due to their specificity and the duration of action they offer from the standpoint of drug delivery. Liposome compositions can be formed, for example, from phosphatidylcholine, dimyristoyl phosphatidylcholine, dipalmitoyl phosphatidyl-choline, dimyristoyl phosphatidylglycerol, or dioleoyl phosphatidylethanolamine. Numerous lipophilic agents are commercially available, including LIPOFECTIN® (Invitrogen/Life Technologies, Carlsbad, Calif.) and EFFECTENE™ (Qiagen, Valencia, Calif.).

Compositions/formulations additionally can contain other adjunct components such as, for example, lubricants, preservatives, stabilizers, wetting agents, emulsifiers, salts for influencing osmotic pressure, buffers, colorings, flavorings, and aromatic substances.

Dosing of compositions for administration to a subject typically is dependent on the severity and responsiveness of the virus infection to be treated or prevented, with the course of treatment lasting, In some example embodiments, from several days to several months, or In other example embodiments until eradication of the infection is affected or a diminution of the symptoms is achieved. One of ordinary skill in the art can routinely determine optimum dosages, dosing methodologies and repetition rates. Optimum dosages can vary depending on the relative potency of individual actives and can generally be estimated based on EC50 found to be effective in in vitro and in vivo animal models.

In some example embodiments, an effective amount can be administered in one or more administrations, applications, or dosages. In one example embodiment, a therapeutically effective amount of a therapeutic compound (i.e., an effective dosage) depends on the therapeutic compounds selected. The compositions can be administered one from one or more times per day to one or more times per week; including once every other day. One of ordinary skill in the art will appreciate that certain factors may influence the dosage and timing required to effectively treat a subject, including but not limited to the severity of the infection with the virus, previous treatments, the general health and/or age of the subject, and other diseases present. Moreover, treatment of a subject with a therapeutically effective amount of the therapeutic compounds described herein can include a single treatment or a series of treatments.

In other example embodiments, dosage, toxicity, and therapeutic efficacy of the therapeutic compounds and/or compositions of the present disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care can be taken to design a delivery system that targets such compounds and/or compositions to the site of infected cells and/or tissue in order to minimize potential damage to uninfected cells and/or tissue and, thereby, reduce side effects.

In some example embodiments, data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured by techniques known in the art, for example, by high performance liquid chromatography.

In other example embodiments, a preliminary dosage for human infection can be inferred using guidelines put forth by the FDA (Guidance for Industry: Estimating the Maximum Safe Starting Dose in Initial Clinical Trials for Therapeutics in Adult Healthy Volunteers Food and Drug Administration, Editor. 2005 (Rockville, Md.), which is herein incorporated by reference in its entirety).

One or more embodiments provides a composition comprising an effective amount of a compound that inhibits PERK activity and/or expression in a cell infected with a virus, wherein the effective amount is sufficient to reduce translation of a nonstructural protein of the virus in the cell.

In some example embodiments, the compound is a small interfering RNA (siRNA).

In one example embodiment, the composition is a pharmaceutical composition as described herein further comprising a pharmaceutically acceptable carrier.

In other aspects, the present disclosure provides a kit.

The compositions (e.g., pharmaceutical compositions) and/or compounds of the present disclosure can be included in a container, pack, or dispenser together with instructions for administration e.g., as one or more component contained in a kit.

The disclosure will be further described in the following examples, which do not limit the scope of the disclosure described in the claims.

### EXAMPLES

### Example 1: Materials and Methods

Cell Culture

Primary human astrocytes and Human Umbilical Vein Endothelial Cells (HUVECs) were obtained from Lonza and maintained in Astrocyte growth medium (AGM) BulletKit (CC-3187 & CC-4123) and Endothelial cell growth medium (EGM)-2 BulletKit (CC-3156 & CC-4176) respectively. Human brain microvascular pericytes were obtained from Sciencell and maintained in Pericyte medium (PM) supplemented with 2% fetal bovine serum (FBS), 1% PM growth supplement, and 1% of penicillin/streptomycin solution. Vero (ATCC CCL-81) and the human glioblastoma cell line, U87MG (ATCC HTB-14) were obtained from ATCC and maintained in Dulbecco's modified minimum essential medium (DMEM) supplemented with 10% fetal bovine serum (FBS), 1% L-glutamine, and 1% penicillin/streptomycin. All cells were maintained at 37° C. with 5% CO₂.

Viruses and Infections

VEEV TC-83, VEEV Trinidad Donkey (TrD) (epidemic subtype I/AB/C), eastern equine encephalitis virus (EEEV) FI93-939, VEEV nsP3-nLuc, and EEEV nsP3-nLuc viral stocks were produced by electroporation of Vero cells with in vitro-transcribed viral RNA generated from molecular clones. VEEV nsP3-nLuc, EEEV FI93-939, and EEEV nsP3-nLuc plasmids were kindly provided by William Klimstra of the University of Pittsburgh. Experiments with VEEV TC-83 were performed under BSL-2 conditions while all others were performed under BSL-3 conditions. All work involving select agents was registered with the Centers for Disease Control and Prevention and conducted at George Mason University's Biomedical Research Laboratory, which is registered in accordance with federal select agent regulations.

For viral infections, cells were plated in 12-well (2.5×105 cells/well) or a 24-well (1.4×105 cells/well) plates and incubated overnight. Next day, cells were infected at the specified multiplicity of infection (MOI). Cells were infected for 1 h at 37° C. and rocked every 15 min to ensure adequate coverage. The cells were then washed with phosphate-buffered saline (PBS), and complete growth medium was added back to the cells. Viral supernatants and cells were collected at various times post-infection for further analysis. To determine viral titers, astrocytes were seeded in a 96-well plate at 15,000 cells per well and allowed to incubate overnight at 37° C. and 5% CO2. Treatments and infections were performed as described above. Supernatants were collected at the indicated time points and stored at −80° C. until use. Viral titers were determined by crystal violet plaque assay using Vero cells as previously described.

RNA Isolation and Quantitative RT-PCR

Total RNA extracted from mock-infected or VEEV TC-83-infected astrocytes was isolated using the RNeasy mini kit (Qiagen) using manufacturer's instructions. Reverse transcription quantitative PCR (RT-qPCR) was performed using the StepOnePlus™ Real-Time PCR System (Life Technologies). TaqMan Gene Expression Assays were used for interferon (IFN)-β (Hs0107758_s1) or 18S (Hs99999901_s1). Fold changes were calculated relative to 18S ribosomal RNA and normalized to mock samples using the ΔΔCt method. RT-qPCR assays to detect viral RNA in astrocytes were performed using Invitrogen's RNA UltraSense™ One-Step Quantitative RT-PCR System using Integrated DNA Technologies primer pairs (forward primer, 5′-TCTGACAAGACGTTCCCAATCA-3′ (SEQ ID NO:4); reverse primer, 5′-GAATAACTTCCCTCCGACCACA-3′) (SEQ ID NO:5) and TaqMan probe (5′-6-carboxyfluorescein-TGTTGGAAGGGAAGATAAACGGCTACGC-6-carboxy-N,N,N′,N′-tetramethylrhodamine-3′; SEQ ID NO:6) for nucleotides 7931 to 8005 of VEEV TC83 as described previously. The absolute quantification was done using StepOne software v2.3 based on the threshold cycle relative to the standard curve. The standard curve was determined using serial dilutions of VEEV TC-83 RNA at known concentrations.

Western Blot Analyses

Protein lysates were collected using Blue Lysis Buffer and analyzed by western blot. The recipe for Blue Lysis Buffer consists of: 25 ml 2× Novex Tris-Glycine Sample Loading Buffer SDS (Invitrogen, Cat # LC2676), 20 ml T-PER Tissue Protein Extraction Reagent (ThermoFisher, Cat # 78510), 200 μl 0.5M EDTA pH 8.0, 2-3 complete Protease Cocktail tablets for 50 ml, 80 μl 0.1M Na₃VO₄, 400 μl 0.1M NaF, 1.3 ml 1M dithiothreitol. Briefly, primary antibodies against capsid of Venezuelan equine encephalitis virus, TC83 (Subtype |A/B) Capsid (antiserum, Goat) (BEI resources, NR-9403), VEEV GP (antiserum, Goat), VEEV nsP2 (kerafast, Cat # 8A4B3), PERK (C33E10) (Cell Signaling, Cat # 3192S), or horse radish peroxidase (HRP)-conjugated β-actin antibody (Abcam, Cat # ab49900) were diluted in 3% milk solution per the manufacturer's recommended dilutions followed by the addition of the appropriate secondary antibody either anti-rabbit HRP-conjugated (Cell Signaling, Cat # 7074), or anti-goat HRP-conjugated antibody. PDVF membranes were imaged on a Chemidoc XRS molecular imager (Bio-Rad) using the SuperSignal West Femto Maximum Sensitivity Substrate kit (ThermoFisher, Cat # 34095).

Transfections

Astrocytes seeded at 2.5×105 cells per well in a 12-well plate or 1.4×105 cells/well in a 24-well plate were transfected with 100 nM SignalSilence® PERK siRNA I (Cell Signaling, Cat # 9024S), or AllStar negative-control small interfering RNA (Qiagen, Cat # 1027280), using 1.2 μl of the DharmaFECT 1 transfection reagent (Dharmacon, Cat # T-2001-02). At 48 h post-transfection, cells were infected with VEEV TC-83 (MOI 5), VEEV TrD (MOI 0.5), or EEEV (MOI 0.5) for 1 h. After infection the medium was replaced with fresh medium. At 3, 6, 9, 12, 18, or 36 hpi supernatants or lysates were collected for downstream analysis. For polyinosinic-polycytidylic potassium salt [poly (I:C)] (Tocris, Cat # 4287) transfection, poly (I:C) was dissolved in water and 4 ug/ml dsRNA was coupled with 2 ul/well lipofectamine 2000 (Thermofisher, Cat # 11668030). Complexed poly (I:C) was transfected into astrocytes that were previously transfected with siNeg or siPERK. SiNeg astrocytes treated with lipofectamine 2000 only was used as a control. At 6 hours post treatment, lysates were collected for downstream analysis.

Cell Viability Assays

Astrocytes were cultured as described above in 96-well white walled plates (Corning, Cat # 3903) and transfected with siRNAs followed by mock infection or VEEV TC-83 infection at a MOI of 5. At 48 hpi, ATP production was measured as an indication of cell viability using the Promega's CellTiter-Glo assay (Cat # G7570).

Nano-Luciferase Assay

VEEV and eastern equine encephalitis virus (EEEV) nsP3-nLuc viruses were used to infect siRNA transfected astrocytes at a MOI of 5. At 18 hpi, luminescence was measured with Promega's Nano-Glo Luciferase Assay system (Promega, N1110). Assays were performed in white-walled, 96-well plates seeded with 15,000-20,000 cells per well following the manufacturer's protocol.

Translation Reporter Assay

VEEV firefly luciferase-expressing translation reporter plasmids were kindly provided by Dr. Klimstra and are described elsewhere. Briefly, VEEV translation reporter encodes firefly luciferase gene (fLuc) fused in frame with 5′,3′ UTR and poly (A) tail from VEEV. The reporter RNA was in vitro transcribed as described previously. Astrocytes that were previously transfected with siNeg or siPERK as described above, were electroporated with 3 μg of the in vitro synthesized host mimic or VEEV reporter RNAs. Electroporated cells were equally divided between 3 wells in 6-well plates and incubated at 37° C. with 5% CO₂. At 2 hours post electroporation cells were washed with PBS and lysed in passive lysis buffer. The intensity of the luminescence was measured by the Dual Luciferase Reporter Assay (Promega) following manufacturer's instructions. Firefly relative light units (RLU) for each sample were normalized to total protein concentration measured by Bradford assay kit (ThermoFisher) and data expressed as RLU/μg of protein.

Statistics

Unless otherwise noted, all statistical analysis was calculated using an unpaired, two-tailed Student's t-test using Graphpad's QuickCalcs software. All graphs contain the mean and standard deviations with an n=3 unless otherwise mentioned.

### Example 2

Silencing PERK Decreases VEEV Replication

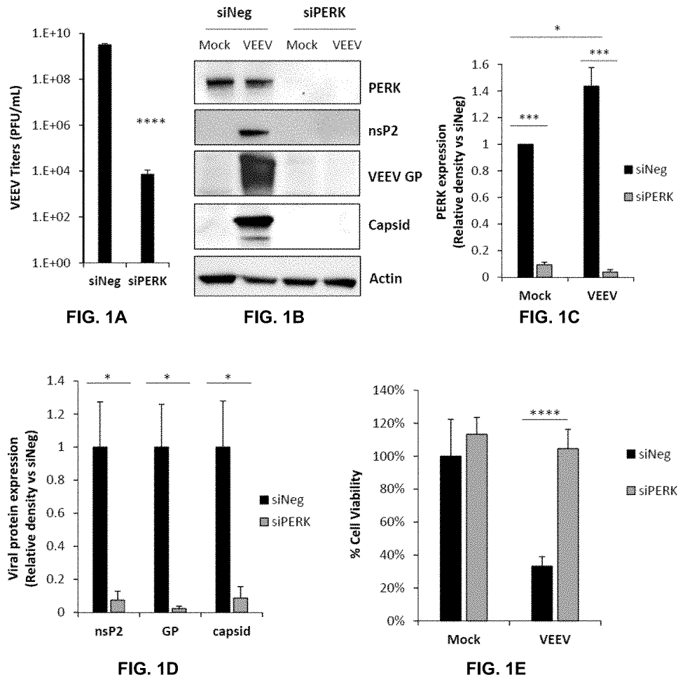

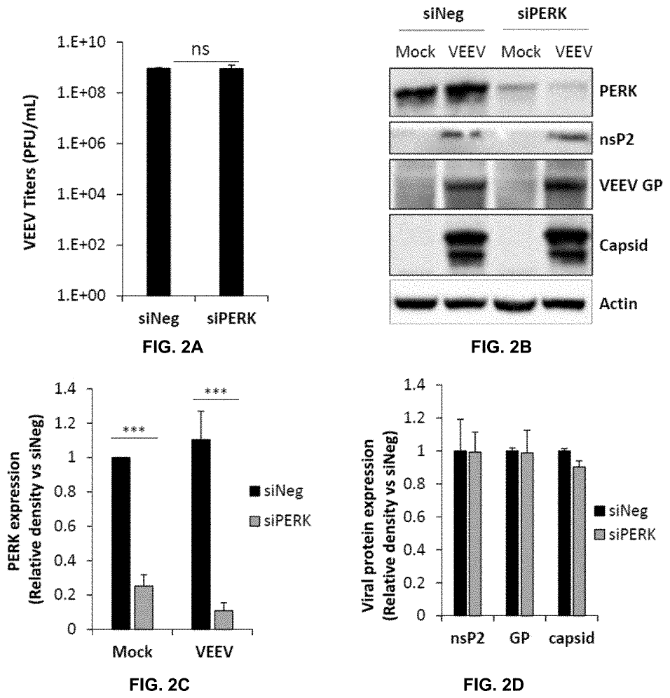

Cells were transfected with siRNA targeting PERK (siPERK) or a negative control siRNA (siNeg) for 48 hours and then infected with VEEV (MOI 5). Viral supernatants were collected, and titers determined via plaque assay. Western blot analysis was performed to determine PERK knockdown and viral proteins. Viral titers were dramatically reduced in siPERK transfected and VEEV-infected primary astrocytes by over 5-log₁₀ (p<0.0001) (FIG. 1A), while there was no significant difference in viral titers between siNeg and siPERK transfected U87MG astrocytoma cells (FIG. 2A). PERK protein was significantly reduced by over 8-fold for both astrocytes and U87MG cells (p<0.001) (FIG. 1B-1C and FIG. 2B-2C). Similarly, a dramatic decrease in VEEV non-structural protein 2 (nsP2), capsid and GP proteins was observed (over 9-fold reduction for each viral proteins) in siPERK transfected and VEEV infected primary astrocytes (FIGS. 1B and 1D), whereas loss of PERK showed no significant difference in viral protein expression in U87MG cells (FIGS. 2B and 2D).

Since blocking PERK signaling had a dramatic decrease in viral titers as well as viral protein expression, we assessed cell viability to ensure that loss of PERK was not negatively impacting cell growth. Only 33% cells were viable in VEEV infected siNeg astrocytes, while no difference in cell viability was observed in VEEV infected siPERK transfected astrocytes or mock infected cells transfected with siPERK (FIG. 1E). These data indicate that blocking PERK signaling drastically impacts VEEV replication in human primary astrocytes and rescues astrocytes from VEEV-induced cell death.

### Example 3

Loss of PERK Reduces VEEV Viral Titers

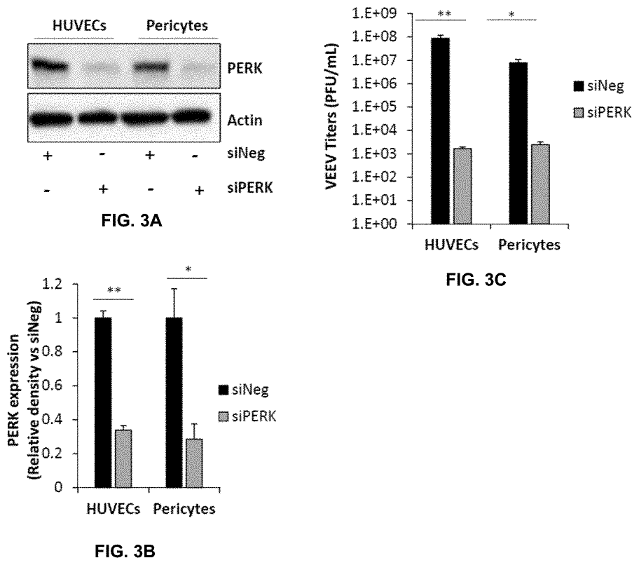

To determine if the loss of PERK is impacts VEEV replication in other cell types, siRNA mediated knockdown of PERK was performed in human primary pericytes and human umbilical vein endothelial cells (HUVECs). At 48 h post transfection lysates were collected for western blot analysis. PERK protein was significantly reduced in cells transfected with siPERK, by over 6-fold for both HUVECs (p<0.01) and pericytes (p<0.05) indicating successful knockdown of PERK (FIGS. 3A and B). VEEV replication was reduced by over 6 log₁₀ in HUVECs (p<0.01) and nearly 4 log₁₀ in pericytes (p<0.02) with siPERK transfection (FIG. 3C). These results indicate that loss of PERK signaling impacts VEEV replication in multiple cell types.

### Example 4

siRNA Knockdown of PERK Reduces Viral Replication

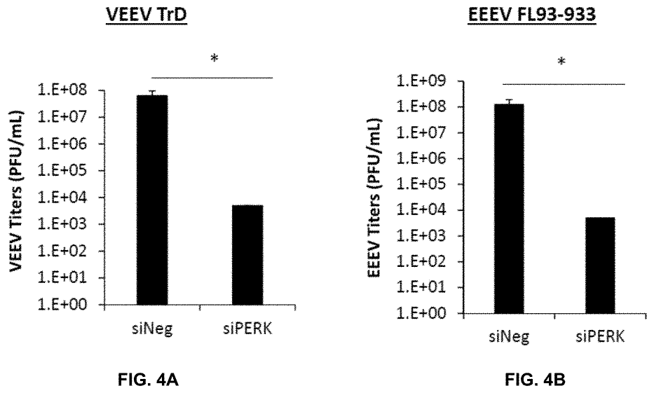

As VEEV TC-83 replication was significantly impacted by the loss of PERK signaling in primary astrocytes, knockdown of PERK was further tested for its effect on VEEV TrD (epidemic subtype I AB/C) and EEEV FL93-939 replication. VEEV (FIG. 4A) and EEEV (FIG. 4B) replication were both significantly reduced by over 4 log₁₀ (p<0.05) following loss of PERK, indicating that silencing PERK significantly impacts viral replication in New World Alphaviruses.

### Example 5

Time Course Analysis of the Impact of PERK on VEEV Replication

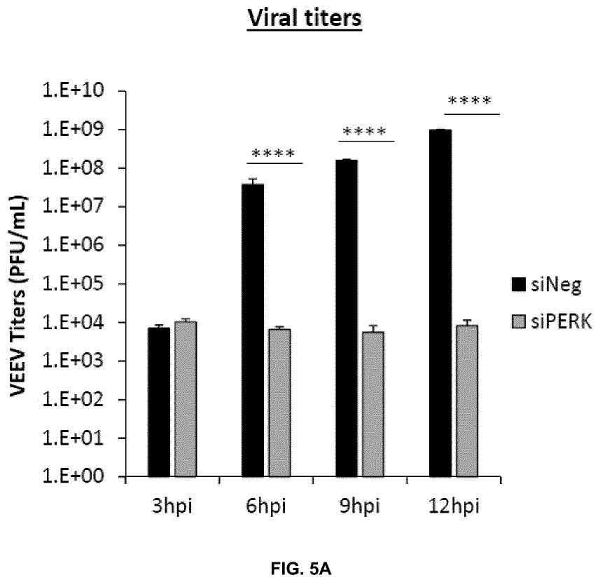

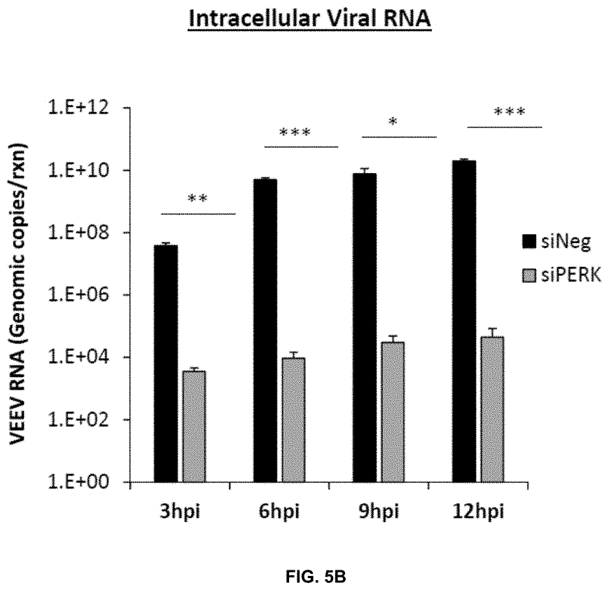

A time course analysis was performed to begin to elucidate the viral life cycle event that is impacted by PERK. Following siPERK or siNeg transfection, astrocytes were infected with VEEV at a MOI of 5, and viral RNA and infectious titer levels determined at 3, 6, 9, or 12 hpi. The viral titers increased over time in siNeg transfected and TC-83 infected astrocytes as expected while in siPERK transfected and VEEV infected cells, viral titers did not increase over time (FIG. 5A). There was a significant and dramatic reduction of viral titers at 6, 9, or 12 hpi in siPERK transfected cells when compared to siNeg transfection, indicating that in the absence of PERK infectious virus is unable to be produced. Similarly, RT-qPCR data showed a significant and dramatic decrease in viral RNA across all time points (FIG. 5B). A small increase in viral RNA in siPERK transfected astrocytes was observed over time, however, there was no significant difference in viral RNA levels at 3 hpi vs 12 hpi. There results indicate that loss of PERK impacts VEEV replication as early as 3 hpi and thus affects an early event in the viral infectious cycle.

### Example 6

VEEV dsRNA is not Synthesized in Cells Lacking PERK

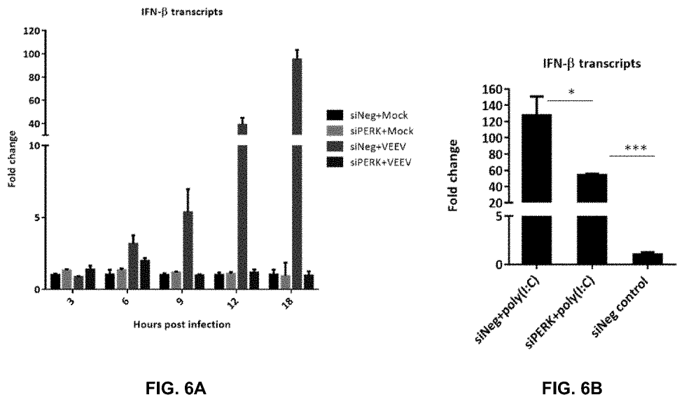

Type1 IFN is induced by the host cells as a first line of defense against viral infections. Since type I IFNs are readily induced in response to viral infection, this initial wave of IFN signaling might serve as an “alarm” signal against viral translation. Therefore, to determine if IFN signaling during early viral infection is impacting viral replication in PERK knockdown cells, siNeg or siPERK transfected astrocytes were mock- or VEEV-infected and total RNA extracted at 3, 6, 9, 12, and 18 hpi. siNeg transfected and VEEV-infected cells induced IFN-β expression beginning at 6 hpi and which a nearly 90-fold change observed at 18 hpi (FIG. 6A). In contrast, siPERK transfected and VEEV infected cells had IFN-β comparable to mock-infected cells at all timepoints, indicating that induction of IFN was not responsible for the dramatic block in VEEV replication observed.

Type 1 IFN is induced by dsRNA synthesized by most viruses during their replication cycle. Since dsRNA is also synthesized by VEEV during its replication cycle, we next performed treatment of siRNA transfected cells with poly (I:C) complexed with Lipofectamine to determine if IFN is capable of being induced in siPERK transfected astrocytes. Our results indicated that with loss of PERK signaling the cells are still able to respond to the dsRNA and induce IFN-β [FIG. 6B, compare siNeg control to siPERK+poly (I:C)]. However, the IFN-β induction was over 70-fold higher in siNeg+poly (I:C) transfected astrocytes compared to those transfected with siPERK+poly (I:C) transfected cells. The reduction in IFN-β gene expression in siPERK transfected and poly (I:C) treated astrocytes may be due to the loss of activation of nuclear factor kappa B (NF-kB) which correlates with reduced IFN-β induction, compared to siNeg astrocytes. These results indicate that cells transfected with siPERK are capable of producing IFN in response to dsRNA. Further, given the lack of IFN induction in siPERK transfected cells following VEEV infection, this suggests that the VEEV dsRNA intermediate is not being produced.

### Example 7

Loss of PERK Signaling Inhibits Translation of Alphavirus Genome

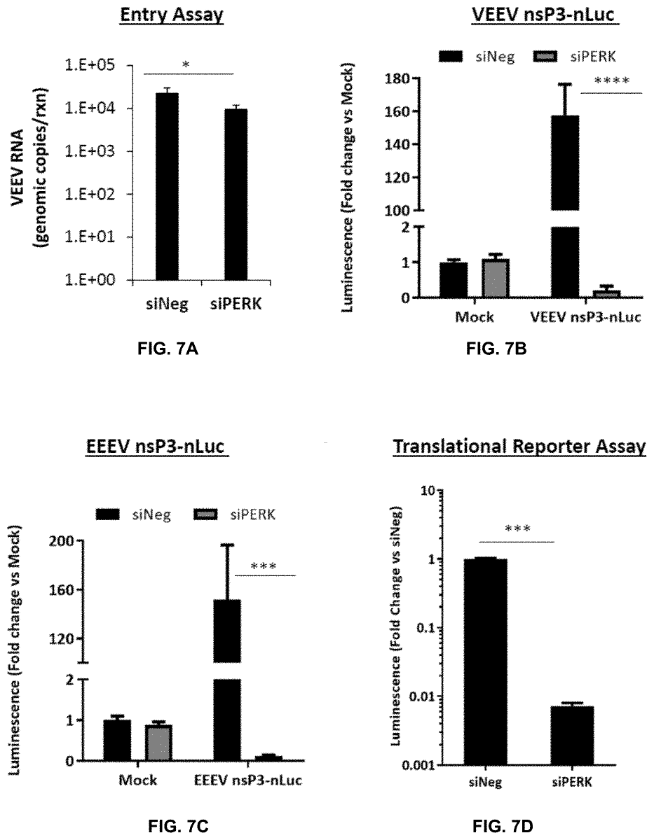

Since the siRNA mediated knockdown against PERK impacted VEEV replication as early as 3 hpi, the impact of PERK on early events including entry and non-structural protein translation was determined. VEEV enters the host cells via receptor-mediated endocytosis in clathrin coated vesicles. Viral entry assays were performed. Briefly, siRNA transfected cells were allowed to remain at 4° C. for 1 h, followed by VEEV infection at 4° C. to allow receptor binding without internalization of the virus so that the infection is synchronized. The temperature was then switched to 37° C. and viral RNA extract at 1 hpi to determine the amount of viral RNA present within the cells. The RT-qPCR analysis showed that VEEV entry was only minimally impacted with the loss of PERK (FIG. 7A), suggesting that this is not the major mechanism by with PERK facilitates VEEV replication.

Alphaviruses undergo translation immediately after uncoating, with nsP1-4 being translated from the incoming viral RNA. To determine if loss of PERK signaling is impacting non-structural protein viral translation, infection of siNeg or siPERK transfected astrocytes with reporter viruses was performed; VEEV nsP3-nLuc or EEEV nsP3-nLuc. The nsP3-nLuc reporter viruses were constructed with the nLuc gene fused in frame with nsP3. When infected, the reporter viruses are able to produce nsP3 fusion protein as an indicator of translation from the incoming genome. The nsP3-nLuc viruses expressed significantly diminished luminescence in siPERK transfected astrocytes. The siNeg transfected and VEEV-nsP3-nLuc virus-infected astrocytes had over 150-fold increase in luminescence over the siPERK transfected cells (FIG. 7B). The EEEV-nsP3-nLuc showed similar results (FIG. 7C). One caveat with this experiment is that viral entry may be contributing to the observed differences. To overcome this limitation and to further confirm the impact of PERK on viral translation, viral translation reporters were used. Astrocytes were transfected with siNeg or siPERK followed by electroporation with translation reporter RNAs. The reporter RNA mimics initial translation of an incoming viral genome and is unable to replicate. The VEEV translational RNA reporter has a cap and a poly-adenylated tail in which the fLuc gene fused with truncated nsP1 was flanked with 5′ and 3′ untranslated regions (UTRs). Furthermore, electroporation of reporter RNAs bypass the entry and fusion steps so that RNAs gets directly delivered into the cytoplasm. The fLuc activity in siPERK transfected and reporter RNAs electroporated astrocytes were significantly lower compared to the siNeg transfected astrocytes (>100-fold reduction) (FIG. 7D). Together these data indicate that PERK is needed for alphavirus nonstructural protein translation.

The above experiments described herein show that PERK is critical for alphavirus replication: it is required for nonstructural protein translation. During the alphavirus replication cycle, as the virus enters cells via receptor mediated endocytosis, the nsPs are translated first, and are essential for subsequent synthesis of genomic and sub-genomic RNAs along with the translation of structural proteins. The data unambiguously shows that PERK impacts an early event during viral infection. In support of this, no IFN-β transcripts were detected in VEEV infected cells transfected with PERK siRNA, suggesting that dsRNA (an alphavirus replication intermediate) was not being produced. This indicates that PERK impacts viral RNA replication or an earlier event. The observation that loss of PERK blocked nsP3-luc reporter virus replication and inhibited production of luciferase from a VEEV translational reporter, narrowed down PERK's activity to translation of nonstructural proteins, which are the first viral proteins synthesized upon infection. Consistent with our data, blocking nonstructural protein translation prevents all subsequent downstream viral steps of the viral infectious program, including viral RNA production, structural protein synthesis and the production of infectious viral particles.

The most well-studied role of PERK is phosphorylation of eIF2α at Ser51, which results in suppression of translation initiation. However, eIF2α is also phosphorylated by protein kinase R (PKR), which senses double-stranded RNA produced during RNA virus infections, including alphaviruses. Alphavirus infected cells display PKR activation and subsequent eIF2α phosphorylation, but translation of the structural proteins from the subgenomic RNA is resistant to eIF2α inhibition. The ability of alphaviruses to overcome eIF2 inhibition has been mapped to a stable RNA hairpin loop in the 26S promoter of the subgenomic mRNA, which enables the ribosome to stall on the correct AUG. Their resistance to eIF2 inhibition could also be linked to alphaviruses not requiring eIF4G for translation. The experiments described herein showing, surprisingly, that PERK is required for alphavirus nonstructural protein translation appears to be paradoxical when put in the context of the traditional role of PERK in suppressing translation.

### Example 8

Loss of PERK Reduces RVFV and ZIKV Viral Titers

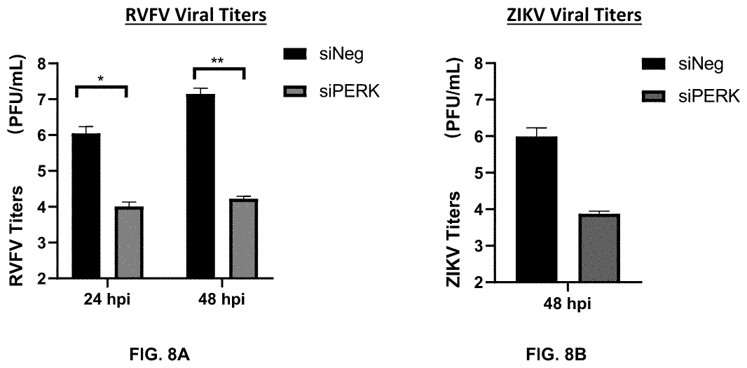

Human astrocytes were transfected with 100 nM of siNeg or siPERK siRNAs. At post 48 h post transfection, cells were infected with RVFV MP12 (MOI 5) or ZIKV MR776 (MOI 5). Supernatants were collected at 24 or 48 hpi for RVFV and at 48 hpi for ZIVK and viral titers determined via plaque assay. There was a significant reduction of RVFV titers at 24 hpi and a more significant decrease at 48 hpi (FIG. 8A). Loss of PERK also resulted in a reduction in ZIVK viral titers at 48 hpi (FIG. 8B) although less significant. These results indicate that PERK is important for the production of infectious viral particles for multiple viruses in human primary astrocytes.

Further, the disclosure comprises additional notes and examples as detailed in the following clauses.

Clause 1. A method for reducing translation of a non-structural protein of a virus in a cell, the method comprising: contacting the cell with an effective amount of a composition that inhibits a protein kinase R (PKR)-like endoplasmic reticulum kinase (PERK) activity and/or expression in the cell, thereby reducing translation of the non-structural protein.

Clause 2. The method of clause 1, wherein the virus is an alphavirus.

Clause 3. The method of clause 1 or 2, wherein the virus is Venezuelan equine encephalitis virus (VEEV).

Clause 4. The method of any one of clauses 1 to 3, wherein the cell is a vertebrate cell.

Clause 5. The method of any one of clauses 1 to 4, wherein contacting the cell comprises contacting the cell in vivo in a subject comprising the cell.

Clause 6. The method of clause 5, wherein the subject is infected or at risk of infection with the virus.

Clause 7. The method of clause 5 or 6, wherein the subject is a human or animal.

Clause 8. The method of any one of clauses 1 to 7, wherein the composition comprises a compound that inhibits the PERK activity and/or expression in the cell.

Clause 9. The method of clause 8, wherein the compound is a small interfering RNA (siRNA).

Clause 10. The method of any one of clauses 1 to 9, wherein the composition is a pharmaceutical composition further comprising a pharmaceutically acceptable carrier.

Clause 11. A method for treating or preventing infection with a virus, the method comprising: administering to a subject an effective amount of a composition that inhibits PERK activity and/or expression in a cell of the subject, thereby reducing translation of a nonstructural protein of the virus in the cell.

Clause 12. The method of clause 11, wherein the virus is an alphavirus.

Clause 13. The method of clause 11 or 12, wherein the virus is Venezuelan equine encephalitis virus (VEEV).

Clause 14. The method of any one of clauses 11 to 13, wherein the subject is a vertebrate.

Clause 15. The method of clause 14, wherein the vertebrate is a human or animal.

Clause 16. The method of clause 14 or 15, wherein the subject is infected or at risk of infection with the virus.

Clause 17. The method of any one of clauses 11 to 16, wherein the composition comprises a compound that inhibits the PERK activity and/or expression in the cell.

Clause 18. The method of any one of clause 17, wherein the compound is a small interfering RNA (siRNA).

Clause 19. The method of any one of clauses 11 to 18, wherein the composition is pharmaceutical composition further comprising a pharmaceutically acceptable carrier.

Clause 20. A method for enhancing viability of a cell infected with a virus, the method comprising:

contacting the cell with an effective amount of a composition that inhibits PERK activity and/or expression in the cell, thereby preventing virus-induced apoptosis in the cell.

Clause 21. The method of clause 20, wherein the virus is an alphavirus.

Clause 22. The method of clause 20 or 21, wherein the virus is Venezuelan equine encephalitis virus (VEEV).

Clause 23. The method of any one of clauses 20 to 22, wherein the cell is a vertebrate cell.

Clause 24. The method of any one of clauses 20 to 23, wherein contacting the cell comprises contacting the cell in vivo in a vertebrate comprising the cell.

Clause 25. The method of clause 24, wherein the vertebrate is infected or at risk of infection with the virus.

Clause 26. The method of any one of clauses 20 to 25, wherein the vertebrate is a human or animal.

Clause 27. The method of any one of clauses 20 to 26, wherein the composition comprises a compound that inhibits the PERK activity and/or expression in the cell.

Clause 28. The method of clause 27, wherein the compound is a small interfering RNA (siRNA).

Clause 29. The method of any one of clauses 20 to 28, wherein the composition is a pharmaceutical composition further comprising a pharmaceutically acceptable carrier.

Clause 30. A composition comprising an effective amount of a compound that inhibits PERK activity and/or expression in a cell infected with a virus, wherein the effective amount is sufficient to reduce translation of a nonstructural protein of the virus in the cell.

Clause 31. The composition of clause 30, wherein the compound is a small interfering RNA (siRNA).

Clause 32. The composition of clause 30, wherein the composition is a pharmaceutical composition further comprising a pharmaceutically acceptable carrier.

What is claimed is:

1. A method for reducing translation of a non-structural protein of a virus in a cell, the method comprising:
contacting the cell with an effective amount of a composition that inhibits a protein kinase R (PKR)-like endoplasmic reticulum kinase (PERK) activity and/or expression in the cell, thereby reducing translation of the non-structural protein.

2. The method of claim 1, wherein the virus is an alphavirus selected from the group consisting of Venezuelan equine encephalitis virus (VEEV), Eastern equine encephalitis virus (EEEV), Western equine encephalitis virus (WEEV), Sindbis virus (SINV), Chikungunya virus (CHIKV), O'Nyong-Nyong virus (ONNV), Ross River virus (RRV), Mayaro virus (MAYV), Semliki Forest virus (SFV).

3. The method of claim 1, wherein the virus is Venezuelan equine encephalitis virus (VEEV) or Eastern equine encephalitis virus (EEEV).

4. The method of claim 1, wherein contacting the cell comprises contacting the cell in vivo in a subject comprising the cell.

5. The method of claim 4, wherein the subject is infected or at risk of infection with the virus.

6. The method of claim 4, wherein the subject is a human or animal.

7. The method of claim 1, wherein the composition comprises a compound that inhibits the PERK activity and/or expression in the cell.

8. The method of claim 7, wherein the compound is a small interfering RNA (siRNA).

9. The method of claim 1, wherein the composition is a pharmaceutical composition further comprising a pharmaceutically acceptable carrier.

10. A method for treating or preventing infection with a virus, the method comprising:
administering to a subject an effective amount of a composition that inhibits PERK activity and/or expression in a cell of the subject, thereby reducing translation of a nonstructural protein of the virus in the cell.

11. The method of claim 10, wherein the virus is an alphavirus.

12. The method of claim 11, wherein the virus is Venezuelan equine encephalitis virus (VEEV) or Eastern equine encephalitis virus (EEEV).

13. The method of claim 10, wherein the subject is a human or animal.

14. The method of claim 13, wherein the subject is infected or at risk of infection with the virus.

15. The method of claim 10, wherein the composition comprises a compound that inhibits the PERK activity and/or expression in the cell.

16. The method of claim 15, wherein the compound is a small interfering RNA (siRNA).

17. The method of claim 10, wherein the composition is pharmaceutical composition further comprising a pharmaceutically acceptable carrier.

18. A composition comprising an effective amount of a compound that inhibits PERK activity and/or expression in a cell infected with a virus, wherein the effective amount is sufficient to reduce translation of a nonstructural protein of the virus in the cell.

19. The composition of claim 18, wherein the compound is a small interfering RNA (siRNA).

20. The composition of claim 19, wherein the composition is a pharmaceutical composition further comprising a pharmaceutically acceptable carrier.
